(19) United States
(12) Patent Application Publication
(10) Pub. No.: US 2015/0177256 A1
(43) Pub. Date: Jun. 25, 2015

(54) TEST STRIP SAMPLE APPLICATION VIDEO SYSTEM

(71) Applicant: Cilag GmbH International, Zug (CH)

(72) Inventors: David ELDER, Inverness (GB); Steven SETFORD, Ross-shire (GB); Ryan WALSH, Douglassville, PA (US); Allan FAULKNER, Ross-shire (GB)

(73) Assignee: Cilag GmbH International, Zug (CH)

(21) Appl. No.: 14/139,437

(22) Filed: Dec. 23, 2013

(51) Int. Cl. G01N 33/66; G01J 1/42; H04N 5/232; H04N 5/225; H04N 5/33

(52) U.S. Cl. CPC G01N 33/66; H04N 5/2257; H04N 5/2256; H04N 5/2253; H04N 5/33; H04N 5/23293; G01J 1/4204

(57) ABSTRACT

An analyte meter having a test strip port includes a camera which is configured to transmit digital images of a test strip in the test strip port for display to facilitate simultaneous application of a sample to the test strip.

### TECHNICAL FIELD

This application generally relates to the field of blood analyte meters and more specifically to portable blood glucose meters that are equipped to magnify a test strip used in conjunction with the meter so that users may clearly view sample application onto the test strip.

### BACKGROUND

Blood glucose measurement systems typically comprise an analyte meter that is configured to receive a biosensor, usually in the form of a test strip. Because many of these systems are portable, and testing can be completed in a short amount of time, patients are able to use such devices almost anywhere during the normal course of their daily lives without significant interruption to their personal routines. A person with diabetes may measure their blood glucose levels several times a day as a part of a self management process to ensure glycemic control of their blood glucose within a target range. A failure to maintain target glycemic control can result in serious diabetes-related complications including cardiovascular disease, kidney disease, nerve damage and blindness.

There currently exist a number of available portable electronic devices that can measure glucose levels in an individual based on a small sample of blood applied to a small glucose test strip. To provide the sample, a person is required to prick their finger in order to apply the blood sample onto the test strip using a lancet or similar implement. Due to the economics of test strip fabrication and many users' desire for reduced-dimension devices, the test strips may be fairly small and difficult to see for some users and, in other cases, the visual acuity of the subjects themselves may create an impediment to reliable or sufficient sample application. It would be advantageous to provide glucose test meters with features to enable users to clearly view the sample chamber on a test strip for purposes of application.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1A is a diagram of an exemplary test strip-based blood analyte test meter;

FIG. 1B is a diagram of an exemplary processing system of the test strip-based blood analyte test meter of FIG. 1A;

FIG. 2A is a diagram of the exemplary test strip-based blood analyte test meter of FIG. 1A in a slide-open position;

FIG. 2B is a side view of the exemplary test strip-based blood analyte test meter of FIG. 2A;

FIG. 2C is a side view of an exemplary test strip-based blood analyte test meter having a tiltable display screen and camera; and

FIG. 3 is a flow chart of an exemplary method of operating the exemplary test strip-based blood analyte test meter of FIG. 1A.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “patient” or “user” refer to any human or animal subject and are not intended to limit the systems or methods to human use, although use of the subject invention in a human patient represents a preferred embodiment.

The term “sample” means a volume of a liquid, solution or suspension, intended to be subjected to qualitative or quantitative determination of any of its properties, such as the presence or absence of a component, the concentration of a component, e.g., an analyte, etc. The embodiments of the present invention are applicable to human and animal samples of whole blood. Typical samples in the context of the present invention as described herein include blood, plasma, red blood cells, serum and suspension thereof.

The term “about” as used in connection with a numerical value throughout the description and claims denotes an interval of accuracy, familiar and acceptable to a person skilled in the art. The interval governing this term is preferably +10%. Unless specified, the terms described above are not intended to narrow the scope of the invention as described herein and according to the claims.

In brief, the system disclosed herein includes a test meter that employs a camera and an illumination source to provide a live video image of a test strip to allow for easier blood sample application onto the test strip. The test meter is activated by detecting insertion of the test trip into a test strip port of the meter. In one embodiment, the meter is placed in a video capture mode by moving at least the camera, or the camera and a display screen, into a predetermined position. The user then views the test strip on the display screen while applying a sample. The invention described herein is equally applicable to patients with other conditions requiring regular self-monitoring of analytes in biological fluids other than glucose.

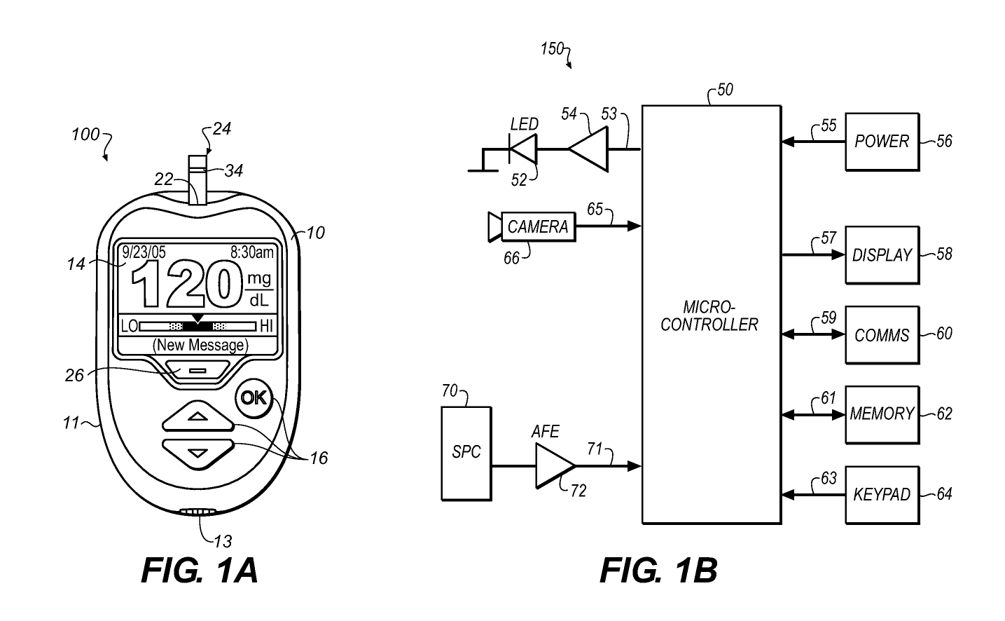

With reference to FIGS. 1A-1B there is illustrated an analyte measurement system 100 that includes an analyte (or test) meter 10. The analyte meter 10 is defined by a housing 11 having an interior that is sufficiently sized to retain a data management unit 150 (FIG. 1B), the housing having a test strip port 22 for receiving a test strip 24. According to one embodiment, the analyte meter 10 may be a blood glucose meter and the test strip 24 is provided in the form of a glucose test strip 24 inserted into the test strip port 22 for performing blood glucose measurements. The analyte meter 10 according to this embodiment further includes a plurality of user interface buttons, or keypad, 16, 26, and a display 14, each disposed on a front facing side of the housing 14, and a data port 13 disposed on one side of the housing opposite the test strip port 22, as illustrated in FIG. 1A. A predetermined number of glucose test strips may be stored in the housing 11 and made accessible for use in blood glucose testing. The plurality of user interface buttons 16 can be configured to allow the entry of data, to prompt an output of data, to navigate menus presented on the display 14, and to execute commands. Output data can include, for example, values representative of an analyte concentration that are presented on the display 14, or image data transmitted from a built-in (or integral) camera 66. User inputs may be requested via programmed prompts presented on the display 14, and a user's responses thereto may initiate command execution or may include data that may be stored in a memory module of the analyte meter 10.

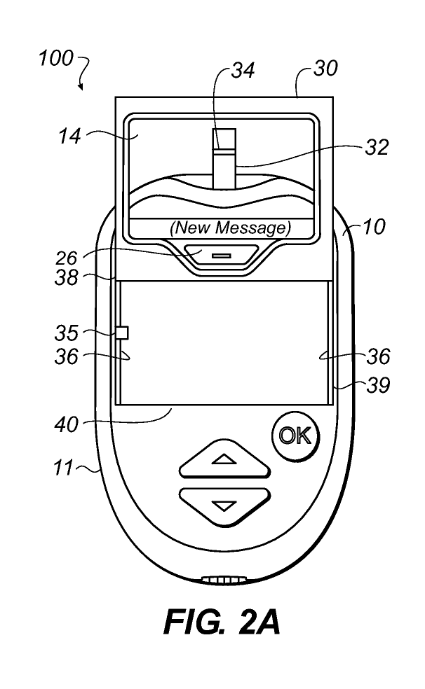

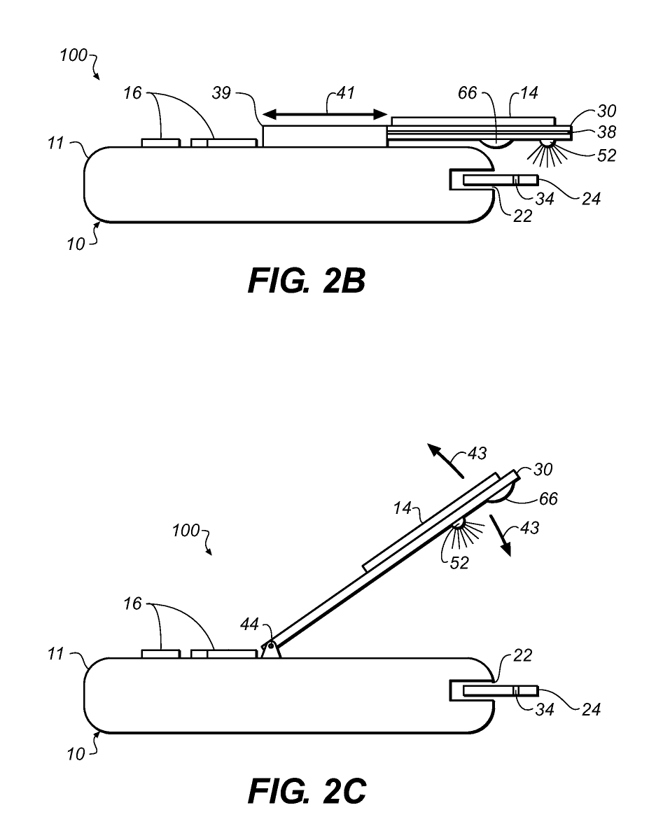

Specifically, and according to this exemplary embodiment, the user interface buttons 16 include markings, e.g., up-down arrows, text characters “OK”, etc, which allow a user to navigate through the user interface presented on the display 14. Although the buttons 16 are shown herein as separate switches, a touch screen interface on display 14 with virtual buttons may also be utilized. As discussed herein, the display 14 may comprise a movable type of display, such as a sliding display (FIGS. 2A-2B) or a display that may be tilted (FIG. 2C). The built-in camera 66, which may be selectively activated by a user or the test meter itself, transmits captured images to the display 14. Preferably, the camera 66 enables real-time video (in-motion) images for transmission to the display 14. The display 14 may be toggled, via button/switch 26, between a mode displaying, for example, analyte measurements results and related information, as exemplified in FIG. 1A, or the display 14 may be toggled in a viewing or video capture mode wherein transmitted video images from the camera 66 are displayed thereon, as shown in FIG. 2A. The in-motion images present a live view of video image data captured by the camera 66. As described below, the camera 66 may be mounted on a portion of the test meter 10 such that the imaging axis of the camera 66 is substantially aligned with a test strip 24 inserted in the test strip port 22 and more particularly, the sample chamber of the inserted test strip 24. As discussed herein, the camera 66 can be fixedly mounted to provide this alignment or the camera may be configured for movement to deploy the camera 66 and/or the display 14 into an operative viewing position.

The electronic components of the glucose measurement system 100 can be disposed on, for example, a printed circuit board situated within the housing 11 and forming the data management unit 150 of the herein described system. FIG. 1B illustrates, in simplified schematic form, several of the electronic sub-systems disposed within the housing 11 for purposes of this embodiment. The data management unit 150 includes a processing unit 50 in the form of a microprocessor, or microprocessor block, a microcontroller, an application specific integrated circuit (“ASIC”), a mixed signal processor (“MSP”), a field programmable gate array (“FPGA”), or a combination thereof, and is electrically connected to various electronic modules included on, or connected to, the printed circuit board, as will be described below. In one embodiment, the processing unit may comprise a microcontroller such as a model STM32F4 series manufactured by ST Microelectronics of Geneva, Switzerland. The camera 66 may be electrically connected to the processing unit 50. For example and according to this exemplary embodiment, the camera 66 is connected to the processing unit 50 via a circuit board interface which may comprise a plug-in type of interface. An illumination device or source 52, such as a single LED controlled by an illumination drive circuit 54, is connected to microcontroller 50 over interface 53, may be used to provide sufficient illumination to permit viewing of images that are captured by the camera 66. Though a single LED is shown, the choice of light source can alternatively include an incandescent bulb, an array of LEDs, or other suitable illumination source as appropriate. In addition, the LEDs may generate a visible light (e.g., white light or colored light), infrared, particularly for use in low-light conditions, or a combination thereof.

The processing unit 50 may be electrically connected to the test strip port connector (“SPC”) circuit 70 positioned in the test strip port 22 via an analog front end sub-system 72. The analog front end 72 is electrically connected to the SPC 70 during blood glucose testing. To measure a selected analyte concentration, the SPC 70 is configured to detect a resistance or impedance across electrodes of the analyte test strip 24 having a blood sample disposed in the sample chamber 34 therein, using a potentiostat, and converts an electric current measurement into digital form for presentation on the display 14, typically in units of mg/dL. The processing unit 50 can be configured to receive input from the SPC 70 via analog front end circuit 72 over an interface 71 and may also perform a portion of the potentiostat function and the current measurement function.

The test strip 24 can be in the form of an electrochemical test strip for measuring a glucose concentration, or other analyte appropriate for monitoring of a biological condition. The test strip 24 is defined by one or more nonporous non-conducting substrates, or layers, onto which one or more electrodes, or conductive coatings may be deposited. These electrodes may function as working electrodes, reference electrodes, counter electrodes or combined counter/reference electrodes. Additional non-conducting layers may be applied in order to define the planar dimensions of the electrode structure(s). Test strip 24 can also include a plurality of electrical contact pads, where each electrode can be in electrical communication with at least one electrical contact pad. SPC 70 can be configured to electrically interface to the electrical contact pads and form electrical communication with the electrodes. Test strip 24 can include a reagent layer that is disposed over at least one electrode forming part of an electrochemical cell of the test strip 24, including the working electrode. The reagent layer can include an enzyme and a mediator. Exemplary enzymes suitable for use in the reagent layer include glucose oxidase, glucose dehydrogenase (with pyrroloquinoline quinone co-factor, “PQQ”), and glucose dehydrogenase (with flavin adenine dinucleotide co-factor, “FAD”). Enzymes other than those used to determine glucose are also applicable, for example, lactate dehydrogenase for lactate, β-hydroxybutyrate dehydrogenase for β-hydroxybutyrate (ketone body). An exemplary mediator suitable for use in the reagent layer includes ferricyanide, which in this case is in the oxidized form. Other mediators may be equally applicable, depending upon the desired strip operating characteristics, for example, ferrocene, quinone or osmium-based mediators. The reagent layer can be configured to physically transform glucose into an enzymatic by-product and in the process generate an amount of reduced mediator (e.g., ferrocyanide) that is proportional to the glucose concentration. The working electrode can then be used to measure a concentration of the reduced mediator in the form of a current magnitude. In turn, microcontroller 50 can convert the current magnitude into a glucose concentration. An exemplary analyte meter performing such current measurements is described in U.S. Patent Application Publication No. US 2009/0301899 A1 entitled “System and Method for Measuring an Analyte in a Sample”, which is incorporated by reference herein as if fully set forth in this application.

A display module 58, which may include a display processor and display buffer, is electrically connected to the processing unit 50 over the communication interface 57 for receiving and displaying output data, and for displaying user interface input options under control of the processing unit 50. The display interface is accessible via the processing unit 50 for presenting menu options to a user of the blood glucose measurement system 100. User input module 64 may receive responsive inputs from the user manipulating buttons, or keypad 16, which are processed and transmitted to the processing unit 50 over the communication interface 63. The processing unit 50 may have electrical access to a digital time-of-day clock connected to the printed circuit board for recording dates and times of blood glucose measurements and user inputs, which may then be accessed, uploaded, or displayed at a later time as necessary.

An on-board memory module 62, that includes but is not limited to volatile random access memory (“RAM”), a non-volatile memory, which may comprise read only memory (“ROM”) or flash memory, and may be connected to an external portable memory device via a data port 13, is electrically connected to the processing unit 50 over a communication interface 61. External memory devices may include flash memory devices housed in thumb drives, portable hard disk drives, data cards, or any other form of electronic storage device. The on-board memory can include various embedded applications executed by the processing unit 50 for operation of the analyte meter 10, as explained herein. On board or external memory can also be used to store a history of a user's blood glucose measurements including dates and times associated therewith. Using the wireless transmission capability of the analyte meter 10, or the data port 13, as described herein, such measurement data can be transferred via wired or wireless transmission to connected computers or other processing devices.

A communications module 60 may include transceiver circuits for wireless digital data transmission and reception, and is electrically connected to the processing unit 50 over communication interface 59. The wireless transceiver circuits may be in the form of integrated circuit chips, chipsets, and programmable functions operable via processing unit 50 using on-board memory, or a combination thereof. The wireless transceiver circuits may be compatible with different wireless transmission standards. For example, a wireless transceiver circuit may be compatible with the Wireless Local Area Network IEEE 802.11 standard known as WiFi. A transceiver circuit may be configured to detect a WiFi access point in proximity to the analyte meter 10 and to transmit and receive data from such a detected WiFi access point. A wireless transceiver circuit may be compatible with the Bluetooth protocol and is configured to detect and process data transmitted from a Bluetooth hub in proximity to the analyte meter 10. A wireless transceiver circuit may be compatible with the near field communication (“NFC”) standard and is configured to establish radio communication with, for example, an NFC compliant reader device capable of gathering analyte test measurements in proximity to the analyte meter 10. A wireless transceiver circuit may comprise a circuit for cellular communication with cellular networks and is configured to detect and link to available cellular communication towers.

A power supply module 56 is electrically connected to all modules in the housing 11 and the processing unit 50 to supply electric power thereto. The power supply module 56 may comprise standard or rechargeable batteries, or an AC power supply that may be activated when the analyte meter 10 is connected to a source of AC power. The power supply module 56 is also electrically connected to the processing unit 50 over the communication interface 55 such that processing unit 50 can monitor a power level remaining in a battery of the power supply module 56.

In addition to connecting external storage for use by the analyte meter 10, the data port 13 can be used to accept a suitable connector attached to a connecting lead, thereby allowing the analyte meter 10 to be wired to an external device such as a personal computer. Data port 13 can be any port that allows for transmission of data, power, or a combination thereof, such as a serial, USB, or a parallel port.

With reference to FIGS. 2A-2C, there are illustrated embodiments of a test meter 10 comprising a movable display panel 30. According to this embodiment, the camera 66 is positioned on the back of a display panel 30, as seen in FIG. 2B, and is positioned proximate the test strip 24 when the display panel 30 is moved, e.g., by sliding the display panel 30, from a first or home, or stored, position 40 to an extended or deployed operating position along a forward direction, as indicated by the double-sided arrow 41. The display panel 30 may be selectively returned to the home position 40 by sliding the display panel 30 in a reverse direction as further indicated by the double-sided arrow 41. A sliding or associated mechanism for enabling movement of the display panel 30 according to this exemplary embodiment includes peripheral or lateral edges 38 of the display panel 30 that are slidably movable within corresponding grooves 36 provided within guide rails 39 on opposing sides of the groove 36.

The camera 66 transmits an image of the test strip 24 to the display module 58 which generates image data to be displayed on the display 14 for more comfortable viewing of the test strip 24 by a user, as shown in FIG. 2A. In one embodiment, the user may adjust a magnification, or zoom, function of the camera 66 at the display interface to enlarge the image on the display 14 so that the user may more easily apply a blood sample from a prick site in the user's finger to the sample chamber 34 of the test strip 24 while simultaneously viewing the test strip 24 in the display 14. Thus, the user may more easily view and guide his or her finger approaching the sample chamber 34 of the test strip 24 to conveniently apply a bodily fluid sample thereto. For example, the zoom function may be controllable via any of the buttons 16, 26 of the housing 11. One feature of the sliding display 14 is that the display 14 obscures the test strip 24 when fully extended. Thus, the user can view the image of the test strip 24 in the display 14 without being distracted by a direct view of the test strip 24. After the user applies a sample to the test strip 24, the sample is detected and an assay is initiated by the test meter 100 as in the usual course. The camera 66 and the camera view mode (FIG. 2A) of the display 14 may be activated to present live motion, real-time images captured by the camera 66 on the display 14 by the sliding movement of the display panel 30 that is detected by the sensor 35, which may comprise an electrical conductive contact. Alternatively, the camera 66 and the display 14 may be manually activated by use of the button 26. The step of activating the camera 66 may include the simultaneous activation of the light source 52. Preferably, sliding the display panel 30 back to the home position 40 automatically deactivates the camera 66 and the light source 52 and reverts the display 14 back into a measurement mode.

With reference to FIG. 2C, there is illustrated an alternative embodiment of a test meter 10 comprising a movable display and display panel 30. For purposes of clarity, similar parts are herein labeled with the same reference numerals. According to this embodiment, the camera 66 is separately attached to one side of a display panel 30 in which the display and display panel 30 are connected to the meter housing 10 via a hinge 44, enabling the display panel 30 to be tilted and adjusted forward or backward, as indicated by the arrow 43, for pointing, or aiming, the camera 66 at the inserted test strip 24 and more specifically the sample chamber 34 thereof. Similar in operation to the sliding display embodiment of FIGS. 2A-2B, the hinge 44 may include electrical contacts for detecting that the display panel 30 has been tilted into a position for aiming the camera 66 at a test strip 24 inserted into the test strip port 22. Upon detecting such movement of the display panel 30, the camera 66, together with the light source 52, may be activated either automatically or manually.

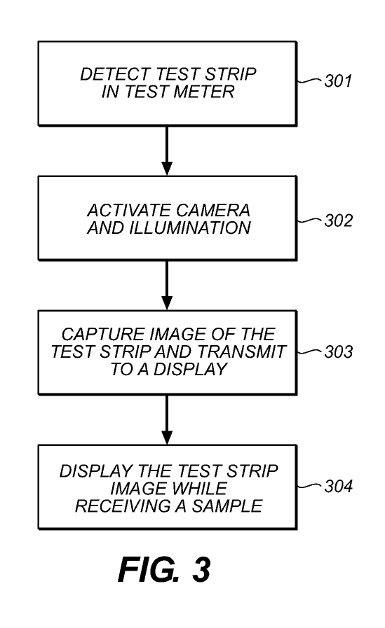

With respect to FIG. 3, there is illustrated an exemplary flow chart of a method of operating the test meter 10. The test meter 10 is activated from a sleep or passive mode, for example, when the test meter 10 detects insertion of a test strip 24 into test strip port 22 at step 301. To activate the camera 66 and the illumination source 52, at step 302, the display panel 30 may be moved by the user such as by sliding the tongue 38 along the groove rails 36, in one direction indicated by arrow 41, or by tilting the display panel 30 about a connecting hinge 44, at step 302, as described herein. It will be readily apparent that other mechanisms, such as motorized assemblies (not shown) can be utilized for moving the display and/or the camera from the home to the strip viewing position. This movement of the display panel 30 activates the camera 66 and the user may further adjust the position of the display panel 30 so that the camera 66 is pointed in a direction enabling best viewing of the test strip 24 on the display 14. This movement can be made manually or the meter can be configured to deploy the display and camera using a motorized arrangement (not shown).

At step 303, the image of the test strip 24 captured by the camera is transmitted to the display 14, which transmission may occur simultaneously with step 302 so that the user may adjust the position of the display panel 30. After positioning the display panel 30 together with the camera 66, the user then may observe the test strip 24 in the display 14 while simultaneously applying a sample to an inlet of the test strip sample chamber 34, at step 304.

The image displayed may be improved over a direct view of the test strip 24 in several ways. One improvement comprises enlarging, or magnifying, the image so that the test strip 24 appears in the display 14 in a larger than actual size for ease of viewing by the user and for more accurate placement of the sample in the sample chamber 34. Another example of improvement of the image may comprise electronically and automatically brightening the image of the test strip 24 if the user is checking blood glucose concentration in a darkened or semi-lighted area, by including an ambient light detector in the test meter 10. As another example, well known image enhancement circuits may be included in the test meter 10 to improve sharpness, edge detection, and overall image brightness and resolution. The light source 52 may be automatically controlled by the microcontroller 50 for brightness by receiving signals from an ambient light detector (not shown). In one embodiment, the light source 52 may comprise an infra red LED for illuminating the test strip 24 in a dark area, such as in a movie theater, without generating light visible to the human eye but which light may be detected by the camera 66. The illumination drive 54 may comprise, for example, a digital-to-analog converter output providing a variable DC voltage between zero (0) and 2V or a pulse-width-modulated (PWM) voltage signal with duty cycle varying between 0 and 100%. The LED may be driven by a DC current varying between zero (0) and the maximum for the LED(s), which may be about 30 mA, or it may be driven by a PWM current signal with varying duty cycle between 0 and 100%.

The camera output signal may be compatible with an 8 to 14-bit parallel camera interface transmitting at a rate of up to about 54 MB/s. Autofocus camera models connectable to a 24-pin slot may be obtained from Sanm Technology Co., Ltd., of Shenzhen, China, which include 300K, 1.3M and 2.0M pixel color digital video CMOS camera modules, convertible to infrared light capture mode, providing magnification options ranging from about 1.5× to about 5×.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible, non-transitory medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Furthermore, the various methods described herein can be used to generate software codes using off-the-shelf software development tools. The methods, however, may be transformed into other software languages depending on the requirements and the availability of new software languages for coding the methods.

### PARTS LIST FOR FIGS. 1A-3

10 analyte meter
11 housing, meter
13 data port
14 display
16 user interface buttons/keypad
22 test strip port
24 test strip
26 button/keypad
30 display panel
32 test strip image, magnified
34 sample chamber
35 display panel sensor
36 grooves
38 tongue
39 guide rails
40 display, home position
41 arrow
43 arrow
44 hinge
50 microcontroller (processing unit)
52 illumination or light source
53 illumination interface
54 illumination drive circuit
55 power supply interface
56 power supply
57 display module interface
58 display module
59 communications module interface
60 communications module
61 memory module interface
62 memory module
63 buttons/keypad interface
64 buttons/keypad module
65 camera interface
66 camera, video
70 strip port connector
71 strip port connector interface
72 analog front end circuit
100 analyte measurement system
150 data management unit
301 step, detect test strip
302 step, activate camera and illumination
303 step, capture/transmit test strip image
304 step, display test strip image while receiving sample

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A test meter comprising:
a meter housing having a test strip port for receiving an analytical test strip inserted therein;
a video camera configured to be aimed toward the test strip for capturing and transmitting video images of the test strip; and
a display screen for displaying the video images of the test strip.

2. The test meter of claim 1, further comprising a zoom function for displaying a magnified version of the video images of the test strip.

3. The test meter of claim 2, including at least one feature to enable the camera to be selectively movable from a first position to a second deployed position in which the camera is aimed at the test strip.

4. The test meter of claim 3, wherein the at least one feature enables the camera to be either slidable or tiltable in moving the camera between the first position and the deployed position.

5. The test meter of claim 4, further comprising a light source for illuminating the test strip.

6. The test meter of claim 5, including at least a second feature to enable the light source to be automatically energized when the camera is deployed.

7. The test meter of claim 6, wherein said at least a second feature comprises a sensor to detect the slidable movement of the camera between the first position and the deployed position.

8. The test meter of claim 5, wherein the light source comprises an infrared light source and the camera comprises an infrared sensitive imager.

9. A test meter comprising:
a test strip port for receiving a test strip;
a video camera and a light source mounted to the test meter, the video camera movable between a first position and a second deployed position; and
a display connected to the video camera, the video camera configured to capture video images of the test strip for transmitting the video images of the test strip to the display.

10. The test meter of claim 9, wherein the camera comprises a circuit for magnifying the video images of the test strip on the display.

11. The test meter of claim 10, wherein the camera is movable from a first stored position to a second deployed position in which the camera is aimed in relation to a sample chamber of an inserted test strip.

12. The test meter of claim 11, wherein the camera is configured to be moved by sliding or rotating the camera into and out of the second deployed position.

13. The test meter of claim 11, in which the camera is driven by a motor from the first position to the deployed position.

14. The test meter of claim 11, including at least one feature that enables a user to move the camera from the first position to the deployed position.

15. The test meter of claim 12, further comprising a light source for illuminating the test strip.

16. The test meter of claim 15, wherein the light source is configured to be automatically energized when the camera is moved to the deployed position.

17. A method of performing a test strip measurement in a test meter having a built-in video camera and a built-in display, the method comprising:
detecting the presence of a test strip that is inserted into a test strip port of the test meter;
activating the built-in video camera to capture video images of the inserted test strip;
transmitting the captured video images of the inserted test strip to the display; and
receiving a sample on the test strip while simultaneously displaying the captured video images on the display.

18. The method of claim 17, wherein the step of transmitting the captured video images includes automatically magnifying the captured video images of the inserted test strip.

19. The method of claim 17, further comprising energizing a light source for illuminating the test strip.

20. The method of claim 19, further comprising automatically activating the light source simultaneously with the step of activating the built-in video camera.

21. A hand-held test meter for use with an analytical test strip in the determination of an analyte in a bodily fluid sample, the hand-held test meter comprising:
a housing;
a microprocessor block; and
a video camera and video camera display,
wherein the microprocessor block and the video camera and video camera display are configured to a capture a real-time image of an analytical test strip inserted into the hand-held test meter and a user's approaching finger and display the real-time image in a magnified format, and
wherein the video camera and video camera display are moveable between a stored position and an operating position.

22. A method for determining an analyte in a bodily fluid sample, the method comprising:
moving a video camera and video camera display of a hand-held test meter from a stored position to an operating position;
inserting an analytical test strip into the hand-held test meter;
displaying a magnified real-time image of the analytical test strip inserted and an approaching user's finger on a display of the video camera and video camera display to guide a user in the application of a bodily fluid sample to the analytical test strip; and
determining an analyte in the bodily fluid sample.
